(19) United States
(12) Patent Application Publication
(10) Pub. No.: US 2015/0305798 A1
(43) Pub. Date: Oct. 29, 2015

(54) PORTABLE ELECTROSURGICAL INSTRUMENT

(71) Applicants: Jon Garito, Hewlett Harbor, NY (US); Shodhan Shetty, North Haven, CT (US)

(72) Inventors: Jon Garito, Hewlett Harbor, NY (US); Shodhan Shetty, North Haven, CT (US)

(21) Appl. No.: 14/700,122

(22) Filed: Apr. 29, 2015

Related U.S. Application Data
(60) Provisional application No. 61/985,546, filed on Apr. 29, 2014.

(51) Int. Cl. A61B 18/12; A61B 18/14

(52) U.S. Cl. CPC A61B 18/1206; A61B 18/1402; A61B 2018/00607

(57) ABSTRACT

A handheld and self-contained electrosurgical instrument includes a self-contained housing, rechargeable batteries, a microprocessor for receiving an external radio frequency signal that defines a selected frequency and current level, a circuit producing an RF signal according to said selected frequency and current level that, when applied to an electrode, performs at least one of cutting, coagulating and combined cutting-coagulating of biological tissue, a radio frequency transformer located within the housing and conductively coupled to the circuit, the transformer for transforming the RF signal according to predefined attributes, and an active monopolar or bipolar electrode for receiving the RF signal from the transformer and contacting biological tissue so as to cut and/or coagulate said biological tissue.

### CROSS-REFERENCE TO RELATED APPLICATIONS

This patent application claims priority to provisional patent application No. 61/985,546 filed Apr. 29, 2014 and entitled “Portable RF Generator.” Provisional patent application No. 61/985,546 is hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### FIELD OF THE INVENTION

The disclosed embodiments relate to the field of electrosurgical devices. More specifically, the disclosed embodiments are directed to electrosurgical devices capable of both monopolar and bipolar configurations.

### BACKGROUND OF THE INVENTION

Electrosurgery is one of many energy based surgical tools and is used on biological tissue to cut, coagulate and tighten skin tissue. Other forms of energy based surgical tools include ultrasonics, lasers, microwaves and cryosurgery. A conventional electrosurgical device may have several modes that depend on the output of the device. Varying the radio frequency (RF) generator output of a conventional electrosurgical device allows the device to cauterize tissue or coagulate blood in a wound. In the cauterizing mode, the electrosurgical electrode generates much more heat than in the cutting mode. When in a cutting mode, a current created when the electrode of the electrosurgical device touches the body incises the tissue.

Electrosurgical electrodes are well known to those in the art. The electrodes may be composed of stainless steel, although some are composed of other alloys such as those containing primarily tungsten, molybdenum, chromium, nickel cobalt, or silver alloy electrodes or metals that have reduces electrosurgical resistance. Efficient cutting at lower power reduces blood loss and leads to much cleaner and less traumatic cuts, resulting in less scar tissue. Use of the ultra-sharp needle also eliminates drag when cutting issue. This “no-touch” technique allows the surgeon a sensitive “feel”, which is a significant benefit when performing extreme microsurgery. When used in the cauterization mode, the ultra-sharp electrode may again be used at relatively lower RF power, thereby eliminating problems associated with excessive heat, such as accidental burns to the patient and/or melting the electrode tip, and with greater control over the direction and location of sparking.

Electrosurgery can be performed using either a monopolar or a bipolar configuration. In a monopolar configuration, the current produced by the surgical device travels through the patient to complete the current cycle, while in a bipolar configuration, the current only passes through the tissue between the two electrodes of the surgical device. The characteristics of the current used in a monopolar configuration is different from the characteristics of the current used in a bipolar configuration. One of the drawbacks of conventional electrosurgical devices is the lack of utility in switching between monopolar and bipolar configurations. The complexity of doing so often leads to surgeons requiring two generators being available at the same time during surgery—one for each configuration. This can be costly and cumbersome in patient rooms and operating rooms with limited budgets and space.

Another well-known drawback of conventional electrosurgical devices is the lack of portability. Typically, electrosurgical devices are powered via a cord that delivers RF energy, which can hamper a surgeons mobility and get in the way of other tools and persons present during surgery. Further, when a cord is used to transmit an RF signal, there may be loss of energy via the cord, which causes excess heat, and which necessitates higher energy at the transmission source. When the cord transmits an RF signal to the surgical site, the RF energy may be dissipated, necessitating higher energy levels at the surgical site. This excessive energy in the form of heat can cause an abundance of unwanted electro-thermal burning or charring of tissue at the surgical site. The need for higher energy producing electrosurgery devices is also inefficient and costly. The stray RF energy also causes interference with other medical electromagnetic devices, such as EKG machines, X-ray units, public address and stereo systems, overhead lighting, and electro-medical chairs and tables. Further, the excessive heat can be disconcerting during delicate surgeries and the cord attached to the device can be cumbersome for the medical professional, as well as his assistants.

A further drawback of conventional electrosurgical devices is the lack of frequency choices. Conventional electrosurgical devices have just one frequency. For example, studies have shown that frequencies in the range of 500 kHz is ideal for coagulation of blood in tissue while other studies have shown that 4 MHz is ideal for the atraumatic cutting of tissue. The lack of such options with regard to frequencies in conventional electrosurgical devices limits their usability and effectiveness during surgery.

Therefore, what is needed is a system and method for improving the problems with the prior art, and more particularly for a more efficient and simple electrosurgical device to facilitate surgery in a user-friendly and safe manner.

### SUMMARY OF THE INVENTION

A handheld and self-contained electrosurgical instrument is provided. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, the electrosurgical instrument includes a self-contained housing that is radio frequency permeable, a plurality of rechargeable batteries located within the housing, the batteries for providing a direct current power source, a microprocessor located within the housing and conductively coupled to the batteries, the microprocessor for receiving an external radio frequency signal that defines a selected frequency and current level, a circuit located within the housing and conductively coupled to the microprocessor, the circuit for producing an RF signal according to said selected frequency and current level of a plurality of selectable frequency and current levels that, when applied to an electrode, performs at least one of cutting, coagulating and combined cutting-coagulating of biological tissue, a radio frequency transformer located within the housing and conductively coupled to the circuit, the transformer for transforming the RF signal according to predefined attributes, and an active monopolar or bipolar electrode located outside a distal end of the housing and conductively coupled to the transformer, the electrode for receiving the RF signal from the transformer and contacting biological tissue so as to cut and/or coagulate said biological tissue.

The foregoing and other features and advantages will be apparent from the following more particular description of the preferred embodiments, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings.

FIG. 1 is an illustration of a top perspective view of the electrosurgical device, in accordance with one embodiment.

FIG. 2 is an illustration of another top perspective view of the electrosurgical device, in accordance with one embodiment.

FIG. 3 is an illustration of a top view of the electrosurgical device, in accordance with one embodiment.

FIG. 4 is an illustration of a side view of the electrosurgical device, in accordance with one embodiment.

FIG. 5 is an illustration of a bottom view of the electrosurgical device, in accordance with one embodiment.

FIG. 6 is an illustration of a top perspective view of the electrosurgical device with battery covers removed, in accordance with one embodiment.

FIG. 7 is an illustration of a bottom perspective view of the electrosurgical device with battery covers removed, in accordance with one embodiment.

FIG. 8 is an illustration of a top perspective view of the electrosurgical device with battery covers and batteries removed, in accordance with one embodiment.

FIG. 9 is an illustration of a bottom perspective view of the electrosurgical device with battery covers and batteries removed, in accordance with one embodiment.

FIG. 10 is an illustration of a bottom perspective view of the electrosurgical device with bottom housing cover removed, in accordance with one embodiment.

FIG. 11 is an illustration of a bottom view of the electrosurgical device with bottom housing cover removed, in accordance with one embodiment.

FIG. 12 is an illustration of a bottom perspective view of the electrosurgical device with bottom housing cover removed and with neutral plate interface, in accordance with one embodiment.

FIG. 13 is an illustration of another bottom perspective view of the electrosurgical device with bottom housing cover removed, in accordance with one embodiment.

FIG. 14 is an illustration of a top perspective view of certain interior components of the electrosurgical device, in accordance with one embodiment.

FIG. 15 is an illustration of a bottom perspective view of certain interior components of the electrosurgical device, in accordance with one embodiment.

FIG. 16 is an illustration of another bottom perspective view of certain interior components of the electrosurgical device, in accordance with one embodiment.

FIG. 17 is an illustration of a top perspective view of the electrosurgical device with front end removed, in accordance with one embodiment.

FIG. 18 is another illustration of a top perspective view of the electrosurgical device with front end removed, in accordance with one embodiment.

FIG. 19 is an illustration of a top perspective view of the electrosurgical device in a disassembled state, in accordance with one embodiment.

FIG. 20 is an illustration of a bottom perspective view of the electrosurgical device in a disassembled state, in accordance with one embodiment.

FIG. 21 is an illustration of a bottom view of the rear printed circuit board of the electrosurgical device, in accordance with one embodiment.

FIG. 22 is an illustration of a side view of the rear printed circuit board of the electrosurgical device, in accordance with one embodiment.

FIG. 23 is an illustration of a top view of the rear printed circuit board of the electrosurgical device, in accordance with one embodiment.

FIG. 24 is an illustration of a bottom view of the front printed circuit board of the electrosurgical device, in accordance with one embodiment.

FIG. 25 is an illustration of a side view of the front printed circuit board of the electrosurgical device, in accordance with one embodiment.

FIG. 26 is an illustration of a top view of the front printed circuit board of the electrosurgical device, in accordance with one embodiment.

FIG. 27 is a block diagram illustrating the main components of the electro surgical device, in accordance with one embodiment.

FIG. 28 is a block diagram of a system including an example computing device and other computing devices.

### DETAILED DESCRIPTION

The following detailed description refers to the accompanying drawings. Wherever possible, the same reference numbers are used in the drawings and the following description to refer to the same or similar elements. While embodiments of the invention may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosed embodiments. Instead, the proper scope of the disclosed embodiments is defined by the appended claims.

In accordance with the embodiments described herein, an electrosurgical instrument is disclosed that overcomes the problems with the prior art as discussed above, by providing a mobile, handheld and battery operated electrosurgical instrument that is simple, ergonomic and easy to use. As an improvement over conventional electrosurgical instruments, the disclosed instrument eliminates the need for cords transmitting the RF energy to the surgical site, thereby reducing clutter and increasing a surgeon's mobility. Also, the lack of a cord in the disclosed electrosurgical instrument reduces or eliminates loss of energy via the cord, and the ensuing excess heat, thereby removing the requirement for higher energy at the transmission source. This results in a more efficient and user friendly electrosurgical instrument. Moreover, the disclosed electrosurgical instrument provides removable and interchangeable front ends—one monopolar and one bipolar—that facilitates the expedient switching between monopolar and bipolar configurations, thereby eliminating the requirement for two generators being available at the same time during surgery. Further, the minimal number of component parts allows for quick and inexpensive fabrication, thereby resulting in an economical electrosurgical instrument. Also, the disclosed instrument is easily maneuverable, easily transportable, inexpensive to manufacture and lightweight in its physical characteristics.

In addition, the disclosed electrosurgical instrument overcomes the problems with the prior art as discussed above by providing an electrosurgical device that is smaller, wireless and portable, which allows said device to be carried by surgeons with ease in their apparel, or clipped to their belts. Further, the disclosed electrosurgical device addresses under-served clinical needs, enables surgical treatment of untreatable patient types, enables a shift of procedures from hospitals to outpatient or doctors' offices, including but not limited to military field surgery and humanitarian disaster rescue and relief. Also, the disclosed electrosurgical device reduces costs of technicians and procedures for set-up, reduces purchasing expenses (as it utilizes less expensive technology), reduces maintenance expenses, decreases healing time at the surgical site, improves surgical performance, reduces operating time for both surgeon and patient, and lowers surgery complication rates. Lastly, the disclosed electrosurgical device provides enhancements of active RF electrode accessories in both bipolar and monopolar operation, which expands surgical functionality.

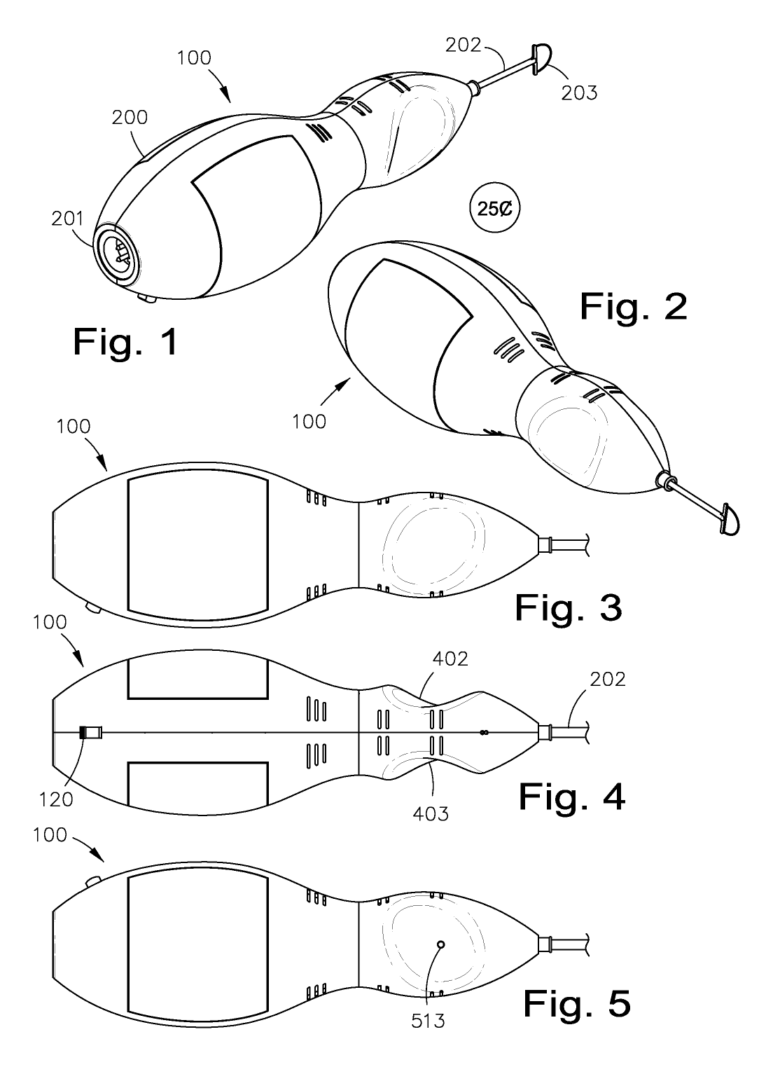

The embodiments of the electrosurgical instrument will be described heretofore with reference to FIGS. 1 through 28 below. FIG. 1 is an illustration of a top perspective view of the electrosurgical device or instrument 100, in accordance with one embodiment, while FIG. 2 shows another top perspective view, FIG. 3 shows a top view, FIG. 4 shows a side view and FIG. 5 shows a bottom view thereof. FIGS. 1-5 depict the housing 200 of the electrosurgical device or instrument 100. The housing 200 is shown as having a bulbous rear portion coupled to a bulbous front portion. The bulbous rear portion is illustrated as having a larger diameter than the bulbous front portion, such that the entire housing 200 is ergonomically shaped to better fit into a surgeon's hand during electrosurgery. To this end, the bulbous front portion of the housing 200 includes two indentations or depressions 402, 403 on opposite sides in the housing, such that the indentations or depressions 402, 403 are shaped to accept a surgeon's fingers. FIGS. 1-5 further depict an electrode 202 including a tip 203 for contacting biological tissue, wherein the electrode 202 is configured for delivering a radio frequency (RF) signal from the electrosurgical device or instrument 100 to a patient's biological tissue via top 203, so as to cut and/or coagulate said biological tissue. In one embodiment, the electrode 202 and/or tip 203 may be removable from the housing and exchanged with other electrodes, as explained further below.

FIG. 1 further shows a terminal 201, which may be a female shaped connection for coupling the device 100 to an external power source, such as a connection to a wall socket that provides an energy source to device 100 to recharge the rechargeable batteries located with the housing 200 of the electrosurgical device or instrument 100. In this embodiment, the external power source may travel through a transformer that converts the external power from AC to DC and steps down the voltage. In another embodiment, at least a portion of the housing 200 is radio frequency permeable, such that external radio frequency waves are able to travel through the housing 200 and enter the inner volume of the housing. In another embodiment, the portion of the housing 200 that is radio frequency permeable is located adjacent to the microprocessor 135 (described below) such that it (or an integrated radio frequency receiver) is able to receive signals from an external source.

FIG. 5 further shows a button or sensor 513 located in the depression 403 of the housing 200. The button or sensor 513 is configured for activating or deactivating the delivery of RF energy from the instrument 100 to the surgical site of the patient during the surgery. The surgeon may depress and release the button or sensor 513 during the surgery when delivering RF energy or pausing. FIGS. 1-5 further show a plurality of vents comprising apertures or other orifices in the housing 200 that allow for ventilation of air from the interior volume of housing 200 to the exterior of the housing.

As shown in FIGS. 1-5, the electrosurgical device or instrument 100 provides a mobile, handheld and battery operated electrosurgical instrument that is completely self-contained and, when charged, does not require any external components to operate for electrosurgery. Further, the relatively small size of the housing (between approximately 4 and 10 inches in length), as well as its ergonomic features, allow for easy handling by a surgeon, and contribute to the device's portability, ease of storage and ease of use. Also, the device 100's lack of a RF delivery cable reduces or eliminates loss of energy via the cable, thereby removing the requirement for higher energy at the transmission source, and resulting in a more efficient and user friendly electrosurgical instrument.

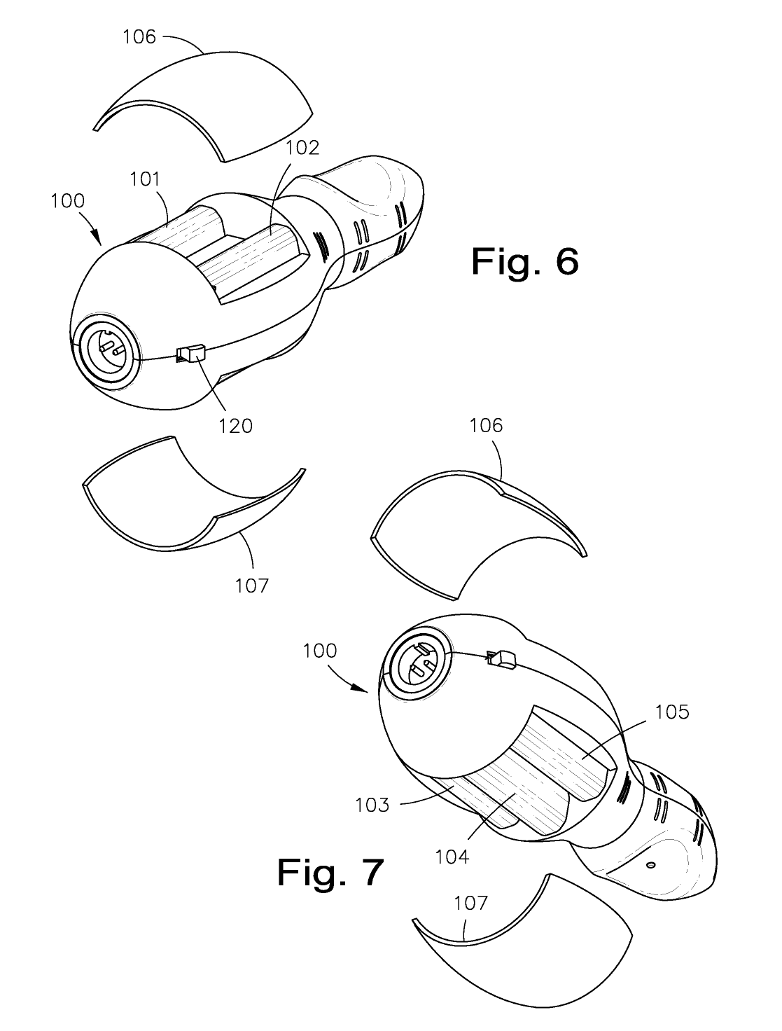

FIG. 6 is an illustration of a top perspective view of the electrosurgical device 100 with battery covers removed, in accordance with one embodiment, while FIG. 7 is an illustration of a bottom perspective view thereof. FIG. 7 shows that the rear bulbous portion of the housing 200 includes a removable top battery cover 106 that, when removed, provides access to the top rechargeable batteries 101, 102, such that no tools are required to remove the rechargeable batteries from the device 100. FIG. 7 also shows that the rear bulbous portion of the housing 200 includes a removable bottom battery cover 107 that, when removed, also provides access to the bottom rechargeable batteries 103, 104, 105, such that no tools are required to remove the rechargeable batteries. Said rechargeable batteries, which provide a direct current power source, may be one or more of lead-acid, nickel cadmium (NiCad), nickel metal hydride (NiMH), lithium ion (Li-ion), Aluminum-ion, and lithium ion polymer (Li-ion polymer) batteries. In one embodiment, each of said rechargeable batteries may provide 2.5 Amps of current at 3.6 Volts. The relatively low amperage and low voltage of the batteries is a result of the proximity of the batteries to the electrode 202 (recall the relatively small size of the housing 200), which reduces loss of energy during transmission within a medium, such as a power cord or other conductor. Any or all of the components of the device 100 that require electricity to operate may be powered by said rechargeable batteries. Note also that although the figures show five batteries, the disclosed embodiments support any number of batteries at any voltage or amps.

FIG. 6 also shows a switch 120, which is an electrical component that controls the electrosurgical device 100. The mechanism of the switch 120 may be operated directly by the fingers of a human operator during use so as to control the device 100. The switch 120 may be used to provide multiple options for the energy source for the device 100. In a first option, the rechargeable batteries may be used as the energy source. In a second option, the power from the outlet may be used as the energy source to provide an alternative DC current source to the device 100, and also to recharge the rechargeable batteries 101-105.

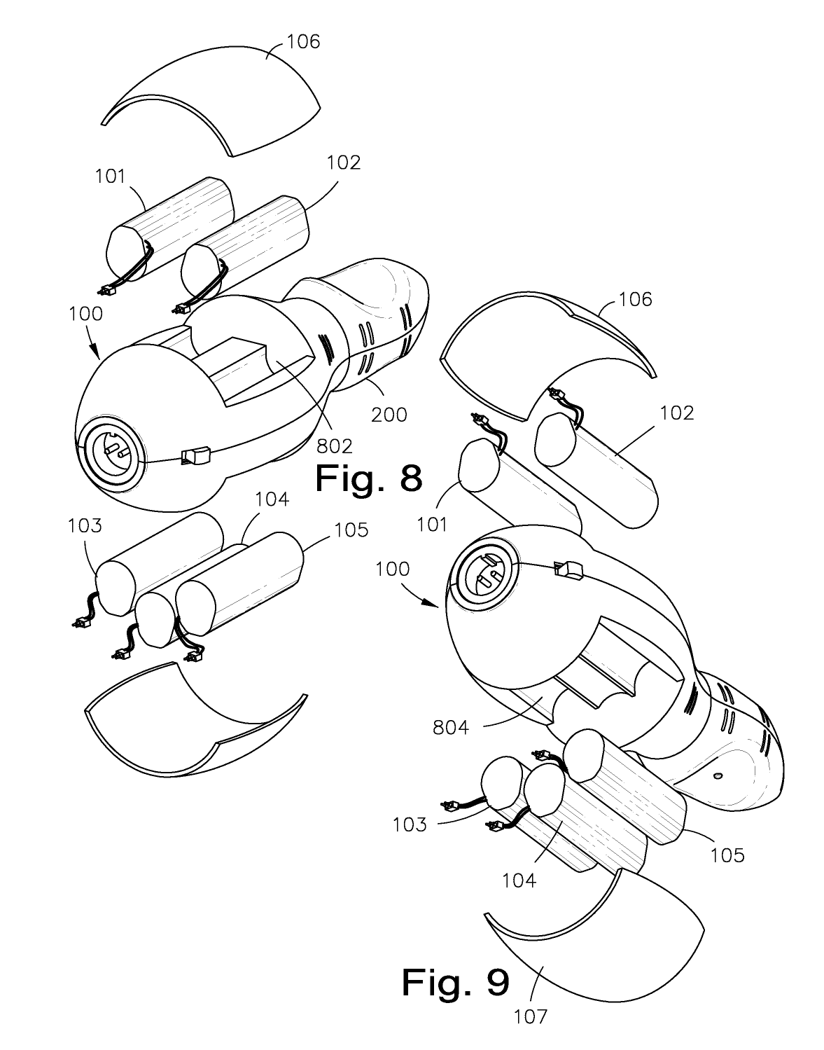

FIG. 8 is an illustration of a top perspective view of the electrosurgical device 100 with battery covers and batteries removed, in accordance with one embodiment, while FIG. 9 is an illustration of a bottom perspective view thereof. FIGS. 8 and 9 show that the top of the rear bulbous portion of the electrosurgical device 100 includes a cavity 802 that is shape for accepting the top rechargeable batteries 101, 102. The cavity 802 may also include contact terminals for contacting the positive and negative terminals of the top rechargeable batteries 101, 102. FIGS. 8 and 9 also show that the bottom of the rear bulbous portion of the electrosurgical device 100 includes a cavity 804 that is shape for accepting the bottom rechargeable batteries 103, 104, 105. The cavity 804 may also include contact terminals for contacting the positive and negative terminals of the bottom rechargeable batteries 103, 104, 105. In one embodiment, the cavity 802 may be sized to provide a friction fit for the top rechargeable batteries 101, 102 and the cavity 804 may be sized to provide a friction fit for the bottom rechargeable batteries 103, 104, 105. In another embodiment, rechargeable batteries 101-105 may each include a wired terminal for conductively coupling the rechargeable batteries 101-105 to a terminal accessible via cavities 802 and 805.

Note that the rechargeable batteries 101-105 may be arranged in a circular pattern within the housing 200. Specifically, as is better illustrated in FIGS. 8 and 9, a cross sectional view taken perpendicular to the longitudinal axis of the housing 200 shows that the rechargeable batteries 101-105 may be arranged in a substantially circular pattern around a midpoint that is approximately located near the longitudinal axis of the housing 200. This allows for weight attributed to the batteries to be concentrated in a desired area—the rear bulbous portion of the housing—so as to facilitate the handheld use of the device 100 by a surgeon.

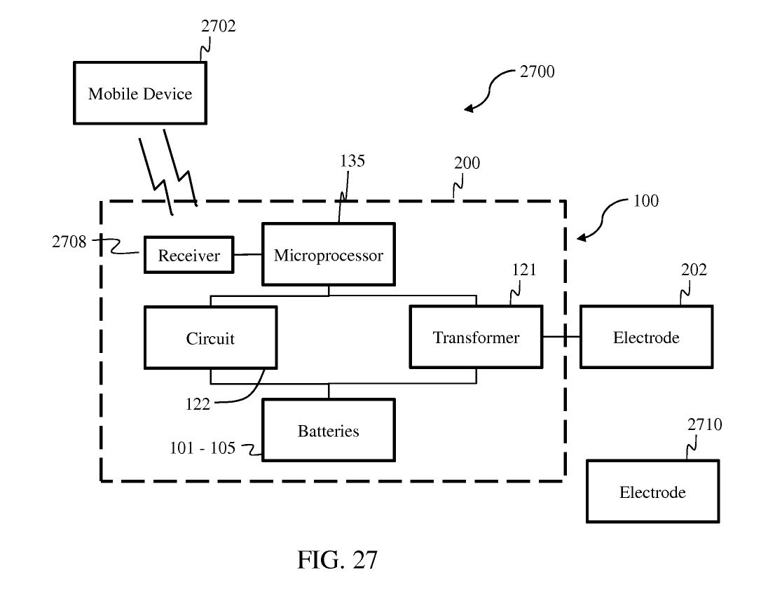

Prior to a discussion of the electronic circuitry of the interior components of the electrosurgical device 100, a more general discussion of the main components of the electrosurgical device 100 is in order. FIG. 27 is a block diagram illustrating the main components of the electrosurgical device 100, as well as a system 2700 for performing electrosurgery, in accordance with one embodiment. The system may include a mobile device 2702 including a display for accepting input from a user and a radio frequency transmitter for transmitting a radio frequency signal that defines a mode. In one embodiment, the mobile device 2702 may be a smartphone, tablet computer, laptop computer or other mobile computing device, such as those described with reference to computing device 2800 below. In one embodiment, the mobile device 2702 may execute an application or other computer program that displays graphical user interface (or an audio interface) for soliciting input from a user. The graphical user interface (or audio interface) allows a user to input a desired mode, frequency modulation or current level for the device 100 via a touchscreen, keypad, voice interface, etc. Consequently, the device 2702 generates a message that represents the data input by the user and transmits said message to the device 100 wirelessly using RF signals.

The radio frequency transmitter of the device 2702 may transmit short-wavelength UHF radio waves in the ISM band from 2.4 to 2.485 GHz, for example. Though device 2702 may use any wireless technology standard for exchanging data over short distances. The mode that is encoded in the transmitted signal may define monopolar mode or bipolar mode for performing electrosurgery or a variety of other modes. Each mode is associated with a modulation, frequency (such as 4 MHz or 500 kHz) and current level of a plurality of selectable modulations, frequency and current levels that, when applied to the electrode 202, performs at least one of cutting, coagulating and combined cutting-coagulating of biological tissue.

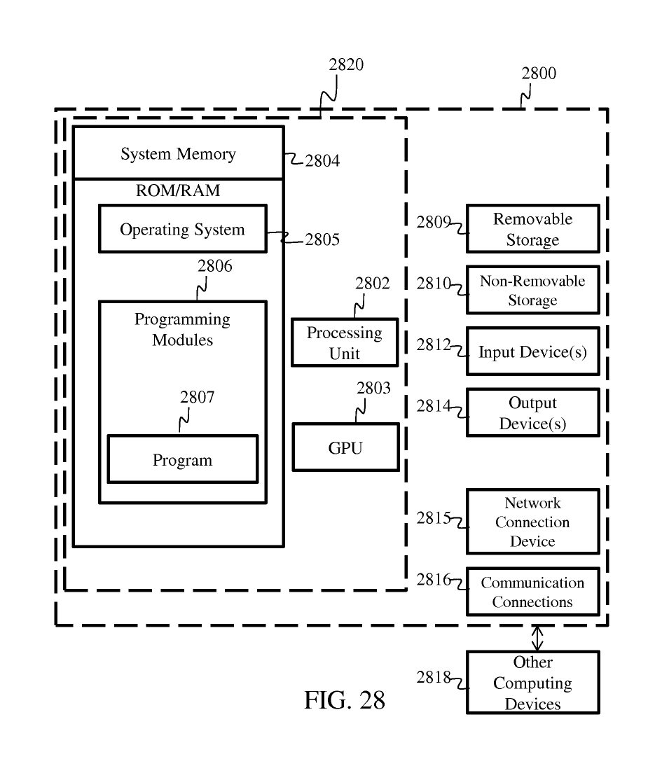

FIG. 27 further shows that the system 100 includes the handheld and self-contained electrosurgical instrument 100 described above, which includes a self-contained housing 200 that is radio frequency permeable and includes at a distal end a terminal 124 for receiving a single electrosurgical electrode 202, a plurality of rechargeable batteries 101-105 located within the housing 200, the batteries for providing a direct current power source, and a microprocessor 135 located within the housing 200 and conductively coupled to the batteries 101-105, the microprocessor 135 for receiving, via receiver 2708, the radio frequency signal from the mobile device 2702, wherein the mode defines a selected mode and/or modulation, frequency and current level. The processor 135 may be a central processing unit, microprocessor, integrated circuit, programmable system on chip device or computing device, as defined below with reference to FIG. 28. In one embodiment, the processor 135 may be a programmable system-on-chip with an antenna 2708 for receiving external radio frequency signals and a wireless circuit. Processor 135 may be configured for receiving and transmitting short-wavelength UHF radio waves in the ISM band from 2.4 to 2.485 GHz, for example. Though processor 135 may use any wireless technology standard for exchanging data over short distances.

The electrosurgical instrument 100 also includes a circuit 122 located within the housing and conductively coupled to the microprocessor 135, the circuit for producing an RF signal according to said selected mode and/or modulation, frequency and current level of a plurality of selectable mode, and/or modulation, frequency and current levels that, when applied to an electrode 202, performs at least one of cutting, coagulating and combined cutting-coagulating of biological tissue.

The circuit 122 may include a clock generator circuit, which may be composed of a crystal oscillator 138 to create an electrical signal with a very precise frequency and a lower power frequency synthesizer chip 139 for generating any of a range of frequencies from the oscillator. The circuit 122 may produce a timing signal for generating a carrier wave or RF signal at the selected frequency and current level of a plurality of selectable frequency and current levels that, when applied to the electrode 202, performs at least one of cutting, coagulating and combined cutting-coagulating of biological tissue.

The circuit 122 may also include a waveform generator for modifying the RF signal or carrier wave according to the selected mode, and/or modulation, frequency and current level of a plurality of selectable mode, and/or modulation, frequency and current levels so as to be suitable for performing electrosurgery using the power source. The waveform generator may generate a repeating RF signal in the analog domain at a variety of wavelengths, frequencies, amplitudes and modulations suitable for performing electrosurgery. The waveform generator may modify the RF signal at a variety of frequencies, amplitudes, etc.

The electrosurgical instrument 100 may also include a radio frequency transformer 121 located within the housing 200 and conductively coupled to the circuit 122 and batteries, the transformer for transforming the modified RF signal or carrier wave according to predefined attributes. The RF transformer 121 converts a given impedance, voltage or current of the modified RF signal or carrier wave to another desired value suitable for performing electrosurgery.

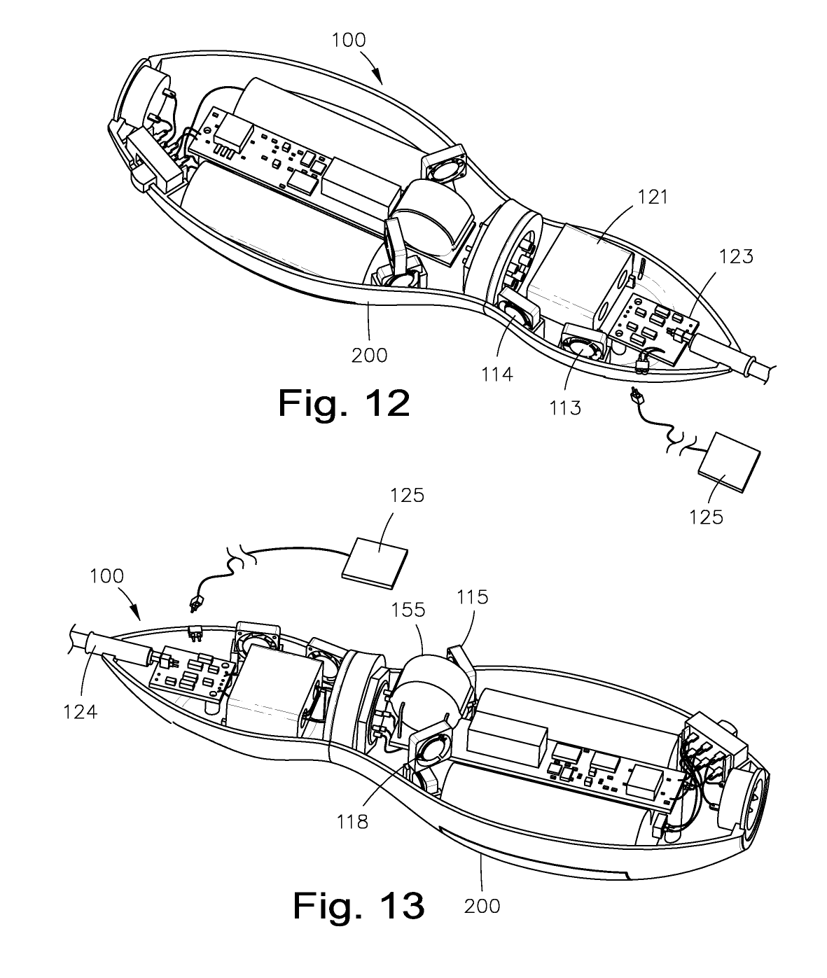

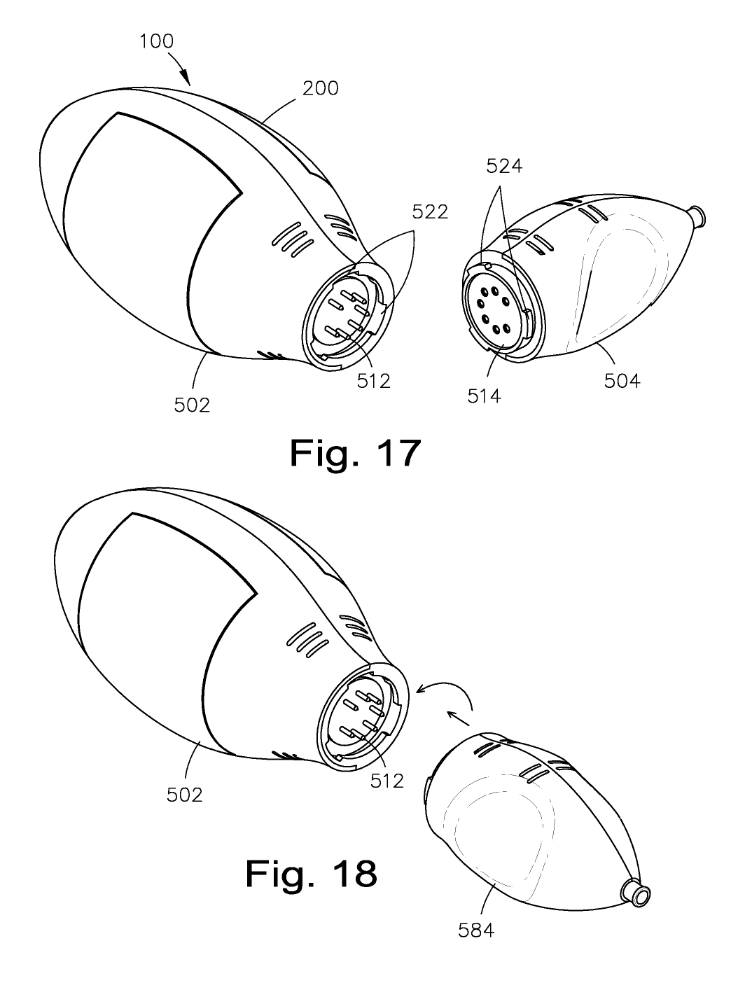

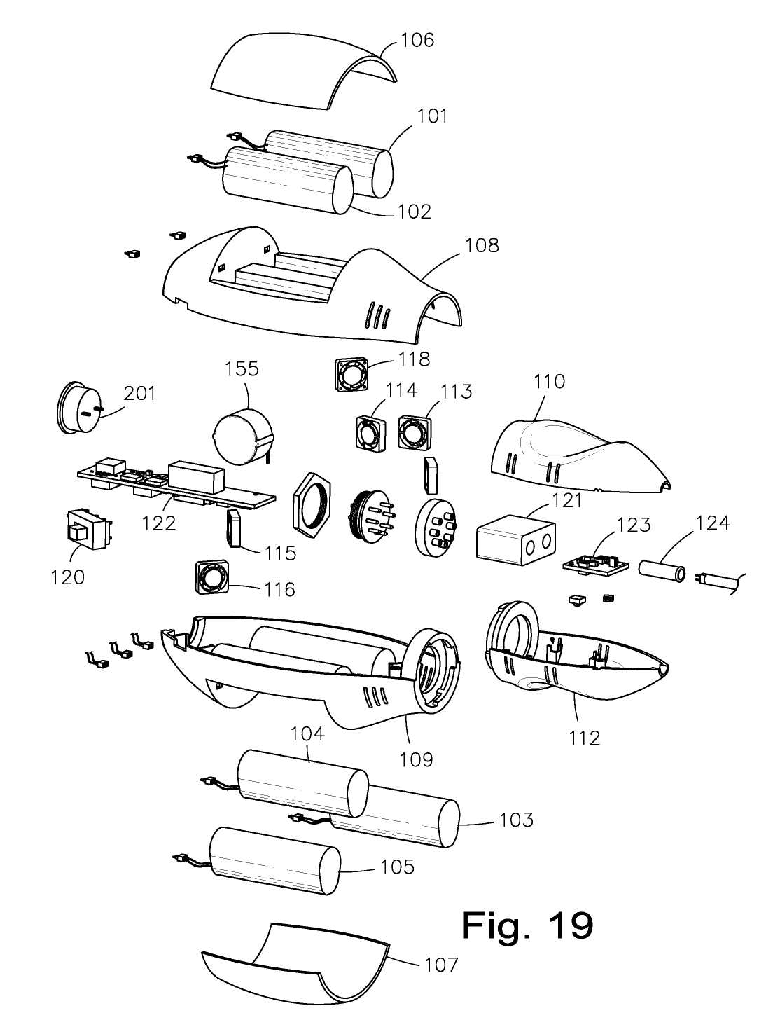

The electrosurgical instrument 100 also includes a removable active monopolar electrode 202 (with a neutral plate 125—see FIG. 12) configured for connecting to the terminal 124 of the housing 200 and thereby conductively coupling to the transformer 121, the electrode for receiving the RF signal from the transformer and contacting biological tissue so as to cut and/or coagulate said biological tissue. The electrosurgical instrument 100 further includes a removable active bipolar electrode 2710 (which is interchangeable with the electrode 202) configured for connecting to the terminal 124 of the housing 200 and thereby conductively coupling to the transformer 121 and contacting biological tissue so as to cut and/or coagulate said biological tissue. Both of the electrodes 202, 2710 may be coupled with or integrated with separate removable front ends, such as front ends 504, 584 (see FIGS. 17-18). These separate removable front ends could be autoclavable, as well as configured for sterile use and may be provided in a sterile bag, and include disposable characteristics. Thus, for example, the monopolar configuration would include the electrode 202, and neutral plate 125 as a separate removable front end in a sterile bag. The surgeon may then remove the separate removable front end from the sterile bag, connect it to the rear bulbous portion, use the front end on the patient during surgery, remove it from the rear bulbous portion, and dispose of the front end after use. The bipolar configuration would be utilized in a similar or identical manner.

In an alternate embodiment, the selectable mode, and/or modulation, frequency and current levels are not received from external source (via the mobile device 2702) but rather from the user entering said data via an interface in the device 100. The interface may comprise one or more of buttons, keys, LEDs, touchscreen, touchpad, display, microphone, movement sensors, etc. located on the exterior of the housing 200 of device 100. In this alternate embodiment, the interface is utilized by the user to enter the selectable mode, and/or modulation, frequency and current levels via touches on the interface, via gestures/movements or via voice command. Further, said display and/or LEDs may further display data such as the selectable mode, and/or modulation, frequency and current levels of the device 100, as well as the charge state of the batteries and other well-known status data that may be displayed about device 100.

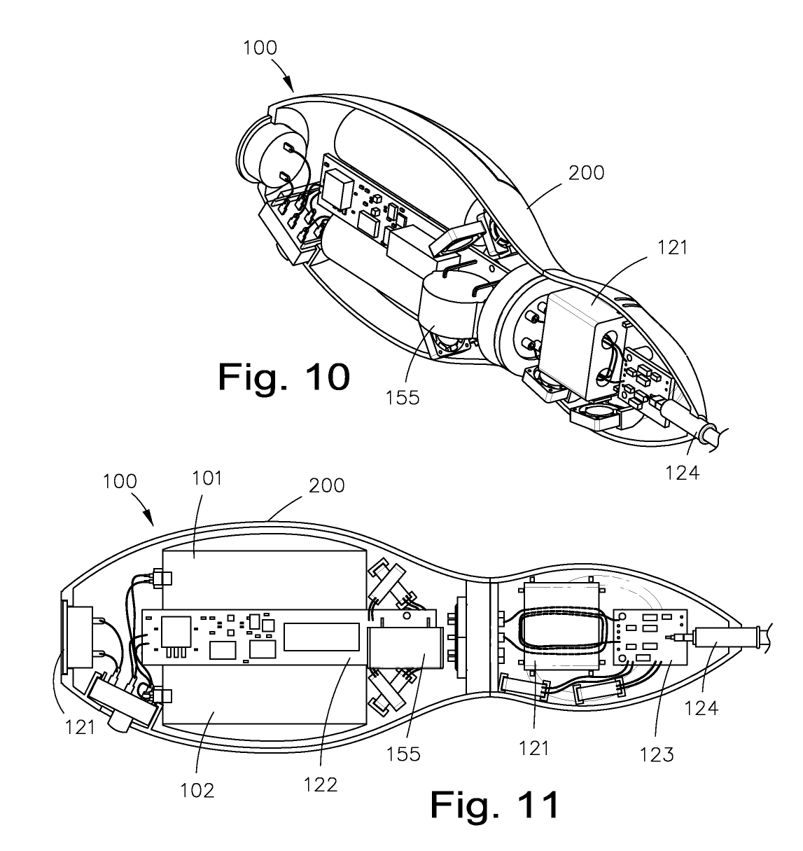

FIG. 10 is an illustration of a bottom perspective view of the electrosurgical device 100 with the bottom housing cover removed, while FIG. 11 is an illustration of a bottom view thereof, FIG. 12 is an illustration of a bottom perspective view thereof and FIG. 13 is an illustration of another bottom perspective view thereof. FIGS. 10-13 show the top rechargeable batteries 101-102 on either side of the printed circuit board (PCB) 122 located in the rear bulbous portion 502 of the electrosurgical device 100. Further, the RF transformer 121 is shown adjacent to and coupled with the printed circuit board (PCB) 123 located in the front bulbous portion 504 of the electrosurgical device 100. Also, the terminal 124 for receiving a single electrosurgical electrode 202, located the distal end of the device 100 is coupled with the PCB 123. The proximity of the electrode 202 to the transformer 121, which eliminates the requirement for any RF energy delivering cable, contributes to the lack of loss of energy during transmission of said energy from the source to the patient's tissue.

FIGS. 12 and 13 further show micro or mini fans 113, 114, 115, and 118 for active cooling, and that may refer draw cooler air into the housing 200 from the outside, expel warm air from inside the housing 200, or move air across a heat sink to cool a particular component of the device 100. FIGS. 12 and 13 also show a high voltage and high current inductor 155, which is used to block AC but allow high voltage high current DC through it. Lastly, FIGS. 12 and 13 further show an external neutral plate 125 conductively coupled with the PCB 123 via a wire, wherein the plate 125 is used in monopolar mode to receive the current that has exited the device 100, so as to complete the circuit.

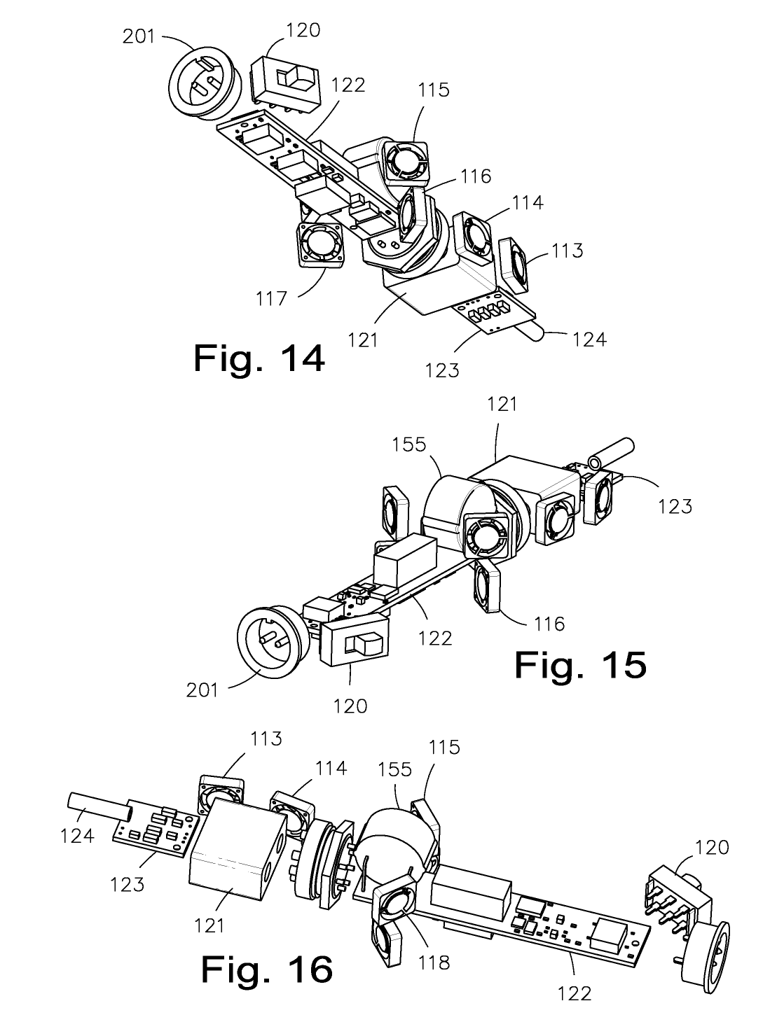

FIG. 14 is an illustration of a top perspective view of certain interior components of the electrosurgical device 100, while FIG. 15 is an illustration of a bottom perspective view thereof and FIG. 16 is an illustration of another bottom perspective view thereof. FIGS. 14-16 show the position of the PCB 122 in relation to the RF transformer 121, the PCB 123 and the inductor 155. The figures also show the switch 120 that controls the electrosurgical device 100. The switch 120 allows the selection of the energy source for the electrosurgical device 100. The selection of the energy source is between the inserted rechargeable batteries or an external power supply via the power outlet. This selected energy source also activates the micro or mini fans 113 through 118.

FIGS. 17 and 18 are illustrations of top perspective views of the electrosurgical device 100 with front end 504 removed, in accordance with one embodiment. Recall that the housing 200 of device 100 comprises a rear bulbous portion 502 and a front bulbous portion 504. In one embodiment, the front bulbous portion 504 (which is designed for monopolar configuration) is removable and replaceable with another front bulbous portion. Note that as described above, portion 504 is autoclavable and may contain disposable characteristics. The mechanism for removably attaching the front bulbous portion 504 to the rear bulbous portion 502 is hereby described.

A plurality of tabs 524 are deposited along the perimeter of the neck of the end of the front bulbous portion 504. The tabs 524 extend radially outward (in relation to the axial centerline) from the neck of the end of the front bulbous portion 504. The tabs 524 are sized and adapted to couple the front bulbous portion to the rear bulbous portion. In the present embodiment, a set of three of tabs 524 is deposited along the perimeter or circumference of the neck. The tabs 524 correspond to the slots 522 along the rim of the rear bulbous portion. The slots 522 are sized so that the outward extending tabs 524 on the perimeter of the neck of the front bulbous portion 504 can enter into the slots 522 when the tabs 524 are aligned with the slots 522.

In one embodiment, the front bulbous portion 504 can be coupled to the rear bulbous portion 502 with a lock created by elongated tabs 524 on the front bulbous portion 504 and the slots 522 on the rear bulbous portion 502. To lock the front bulbous portion 504 to the rear bulbous portion 502, the tabs 524 are received by or inserted into the slots 522 such that the tabs 524 are aligned and correspond to the corresponding slots 522. A force acting towards and along the longitudinal axis of the rear bulbous portion 502 then compresses the two bulbous portions such that the male shaped conductive terminals 512 of the rear bulbous portion 502 are inserted into the female shaped conductive terminals 514 of the front bulbous portion 504, thereby making an electrical connection. After the tabs 524 are inserted into the slots, a force normal to the longitudinal axial centerline rotates the front bulbous portion 504 such that the tabs 524 are no longer aligned with the slots 522. Consequently, the front bulbous portion 504 is coupled to the rear bulbous portion 502. Removing the front bulbous portion 504 from the rear bulbous portion 502 is accomplished by reversing the steps used to couple the two portions.

FIG. 18 further shows a front bulbous portion 584 (which is designed for bipolar configuration) that is also removable and replaceable with front bulbous portion 504. The mechanism for removably attaching the front bulbous portion 584 to the rear bulbous portion 502 is identical to the mechanism for removably attaching the front bulbous portion 504, described above. In one embodiment, either front portion 504, 584 may contain a variety of different electrodes in both monopolar and bipolar configurations. In the monopolar format, the front portion can contain both an indifferent patient plate or wand and the monopolar electrode fully attached in a sterile or non-sterile package. Additionally, each front end portion can contain insulated needles or a large ball in monopolar configuration for skin tightening. In bipolar configuration, the front end portion can be fixed with several insulated needles for penetration of skin in skin tightening procedures. In this configuration, two ball shaped metal electrodes may be used for skin tightening procedures.

In one embodiment, the electrode 202 and/or tip 203 may be removable from front bulbous portions 504, 584 and exchanged with other electrodes, such as loop electrodes, ball electrodes, double wires, straight wires, etc.

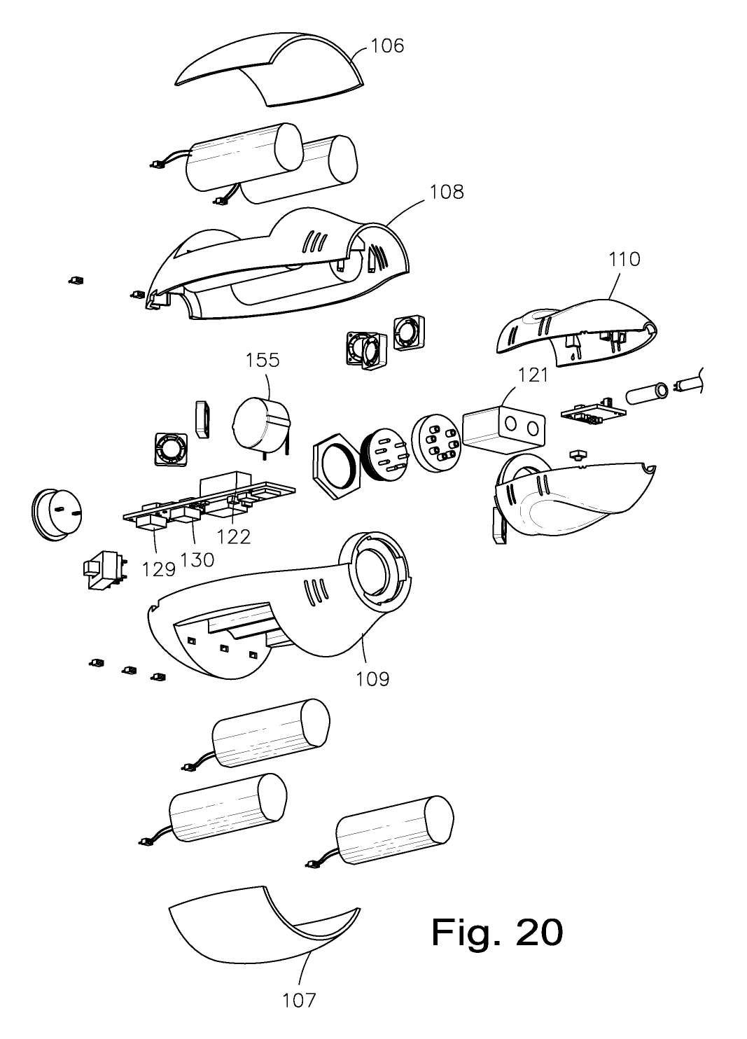

FIG. 19 is an illustration of a top perspective view of the electrosurgical device 100 in a disassembled state, in accordance with one embodiment, while FIG. 20 is an illustration of a bottom perspective view thereof. FIGS. 19-20 show the position of the PCB 122 in relation to the PCB 123 and the inductor 155, as well as the terminal 124 and terminal 201. The figures also show the micro or mini fans 113 through 118. FIGS. 19-20 also show that rear bulbous portion 502 comprises an upper assembly 108 and a bottom assembly 109 while the front bulbous portion 504 comprises an upper assembly 110 and a bottom assembly 112.

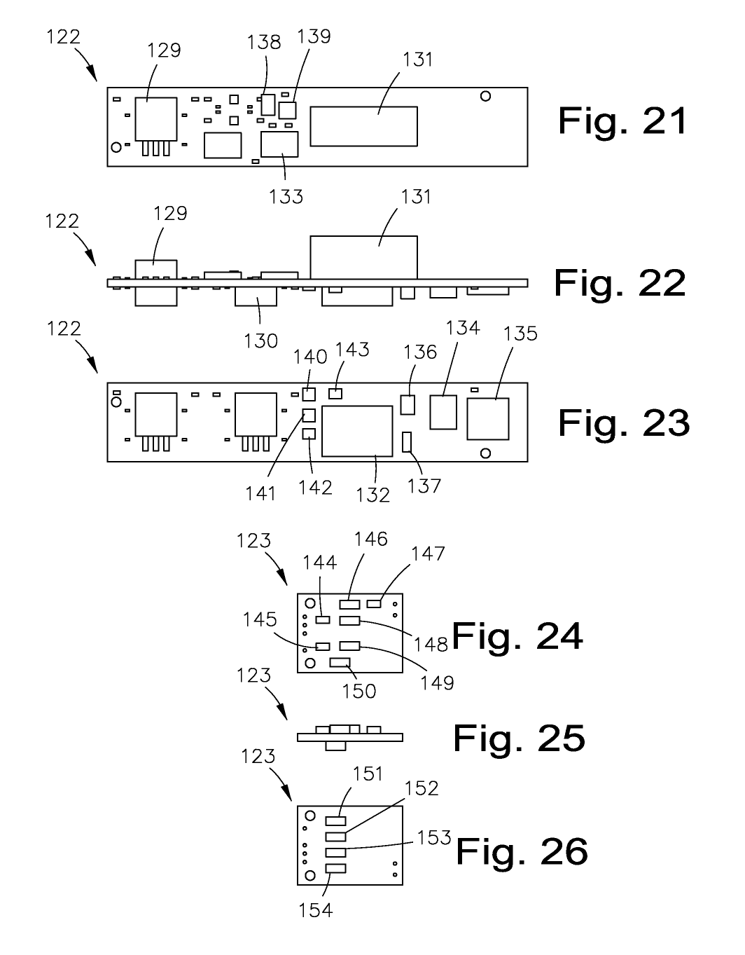

FIG. 21 is an illustration of a bottom view of the rear PCB 122 of the electrosurgical device 100, in accordance with one embodiment, while FIG. 22 is an illustration of a side view thereof and FIG. 23 is an illustration of a top view thereof. The PCB 122 mechanically supports and electrically connects electronic components using conductive tracks, pads and other features etched from copper sheets laminated onto a non-conductive substrate. The PCB 122 can be single sided, double sided, or multi-layer. PCB 122 may include low drop out regulators 129, 130 that can handle high voltage and high current, as well as a crystal oscillator 138 and a low power frequency synthesizer chip 139 (both described above). PCB 122 may further include a high power SMD resistor 131, as well as a diode 133. FIG. 23 shows that the PCB 122 further includes a CMOS controlled SPST switch 140, 141 and a voltage controlled small signal switch 142, as well as a quad 2-input AND gate 143. PCB 122 also includes a power Mosfet 132, high voltage surface mount capacitors 136, 137, a high speed high current gate driver 134 to drive the power Mosfets and the microprocessor 135.

FIG. 24 is an illustration of a bottom view of the front PCB 123 of the electrosurgical device 100, in accordance with one embodiment, while FIG. 25 is an illustration of a side view thereof and FIG. 26 is an illustration of a top view thereof. PCB 123 may include high voltage surface mount capacitors 144 through 150, as well as surface mount resistors 151 through 154 for auto sense detection. The autosense resistors 151-154 provide feedback to the microprocessor 135 (located in the rear section of the handheld) in determining or auto-detecting which front section is connected, i.e., whether the front section is section 504 with a monopolar configuration, whether the front section is section 584 with a bipolar configuration, or whether the front section includes a laser, ultrasonic devices, etc.

FIG. 28 is a block diagram of a system including an example computing device 2800 and other computing devices. Consistent with the embodiments described herein, the aforementioned actions performed by mobile device 2702, as well as processor 135 of device 100 may be implemented in a computing device, such as the computing device 2800 of FIG. 28. Any suitable combination of hardware, software, or firmware may be used to implement the computing device 2800. The aforementioned system, device, and processors are examples and other systems, devices, and processors may comprise the aforementioned computing device.

With reference to FIG. 28, a system consistent with an embodiment of the invention may include a plurality of computing devices, such as computing device 2800. In a basic configuration, computing device 2800 may include at least one processing unit 2802 and a system memory 2804. Depending on the configuration and type of computing device, system memory 2804 may comprise, but is not limited to, volatile (e.g. random access memory (RAM)), non-volatile (e.g. read-only memory (ROM)), flash memory, or any combination or memory. System memory 2804 may include operating system 2805, one or more programming modules 2806 (such as program module 2807). Operating system 2805, for example, may be suitable for controlling computing device 2800's operation. In one embodiment, programming modules 2806 may include, for example, a program module 2807. Furthermore, embodiments of the invention may be practiced in conjunction with a graphics library, other operating systems, or any other application program and is not limited to any particular application or system. This basic configuration is illustrated in FIG. 28 by those components within a dashed line 2820.

Computing device 2800 may have additional features or functionality. For example, computing device 2800 may also include additional data storage devices (removable and/or non-removable) such as, for example, magnetic disks, optical disks, USB or micro-USB drive, or tape. Such additional storage is illustrated in FIG. 28 by a removable storage 2809 and a non-removable storage 2810. Computer storage media may include volatile and nonvolatile, removable and non-removable media implemented in any method or technology for storage of information, such as computer readable instructions, data structures, program modules, or other data. System memory 2804, removable storage 2809, and non-removable storage 2810 are all computer storage media examples (i.e. memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2800. Any such computer storage media may be part of device 2800. Computing device 2800 may also have input device(s) 2812 such as a keyboard, a mouse, a pen, a sound input device, a camera, a touch input device, etc. Output device(s) 2814 such as a display, speakers, a printer, etc. may also be included. The aforementioned devices are only examples, and other devices may be added or substituted.

Computing device 2800 may also contain a communication connection 2816 that may allow device 2800 to communicate with other computing devices 2818, such as over a network in a distributed computing environment, for example, an intranet or the Internet. Communication connection 2816 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 2804, including operating system 2805. While executing on processing unit 2802, programming modules 2806 may perform processes including, for example, one or more of the methods described with reference to processor 135 or mobile device 2702 above. Computing device 2802 may also include a graphics processing unit 2803, which supplements the processing capabilities of processor 2802 and which may execute programming modules 2806, including all or a portion of those processes and methods described with reference to processor 135 or mobile device 2702 above. The aforementioned processes are examples, and processing units 2802, 2803 may perform other processes. Other programming modules that may be used in accordance with embodiments of the present invention may include electronic mail and contacts applications, word processing applications, spreadsheet applications, database applications, slide presentation applications, drawing or computer-aided application programs, etc.

Generally, consistent with embodiments of the invention, program modules may include routines, programs, components, data structures, and other types of structures that may perform particular tasks or that may implement particular abstract data types. Moreover, embodiments of the invention may be practiced with other computer system configurations, including hand-held devices, multiprocessor systems, microprocessor-based DSP-based, FPGA-based or programmable consumer electronics, minicomputers, mainframe computers, and the like. Embodiments of the invention may also be practiced in distributed computing environments where tasks are performed by remote processing devices that are linked through a communications network. In a distributed computing environment, program modules may be located in both local and remote memory storage devices.

Furthermore, embodiments of the invention may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments of the invention may also be practiced using other technologies capable of performing logical operations such as, for example, AND, OR, and NOT, including but not limited to mechanical, optical, fluidic, and quantum technologies. In addition, embodiments of the invention may be practiced within a general purpose computer or in any other circuits or systems.

While certain embodiments of the invention have been described, other embodiments may exist. Furthermore, although embodiments have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the embodiments.

Recall that in accordance with the embodiments described herein, the electrosurgical instrument 100 overcomes the problems with the prior art as discussed above, by eliminating the need for RF-delivering cables, thereby reducing clutter and increasing a surgeon's mobility. Also, the lack of RF-delivering cables between 124 and electrode 202, or similarly for the bipolar configuration, in the disclosed electrosurgical instrument 100 reduces or eliminates loss of energy via the cable, and the ensuing excess heat, thereby removing the requirement for higher energy at the transmission source. This results in a more efficient and user friendly electrosurgical instrument. In traditional electrosurgical units, much of the RF energy is lost in the transmission or conducting cables due to leakage currents, and radiated emissions. This loss of energy can be significant with more of the energy lost due to radiated emissions. Traditional electrosurgical instrument have drawn a certain number of amps at maximum power delivery at a certain wattage. Thus, a certain percentage of the sourced power in traditional electrosurgical units is actually used to deliver the final power to the surgery site. Hence, to apply a certain amount of wattage of power on a patient, a greater amount of wattage of power must be sourced. This large loss of energy in traditional electrosurgery devices is due to various factors, including: (1) efficiency of the power transformers and AC-DC converters in the traditional electrosurgical unit, (2) efficiency of the power amplifiers, (3) loss due to radiated and conducted emissions, and (4) mismatch loss in the traditional electrosurgical unit.

With regard to the disclosed electrosurgical instrument 100, loss due to radiated emissions and mismatch loss is significantly reduced due to the absence of cables for RF energy delivery. In addition, since device 100 used DC batteries, there is no requirement for power transformers, and AC-DC converters, which further reduces the power inefficiencies associated with traditional electrosurgical circuits. Further, the RF power delivery in the device 100 is performed using more efficient Power MOSFETs with a low ON-resistance. Hence, for the same RF energy applied to the patient, the power sourced in the device 100 from the batteries will be about a third of the comparable energy needed in a traditional RF electrosurgical unit. This optimization and increased efficiency achieved in the disclosed device 100 also minimizes thermal dissipation, and hence reduces the cooling requirements for the device 100 by about a fourth of the heat energy dissipated in a traditional RF generator for the same RF energy output delivered. All of the above illustrates the optimization and increased efficiency provided by the disclosed device 100 in comparison with traditional RF electrosurgical units. This reduced energy source and cooling requirements reduces the space required on the housing 200, as well as the overall size of the instrument 100.

Also recall that the disclosed embodiments improve over the prior art by providing a system that does not require an interface on the electrosurgical device 100 itself. The system 2700 include a mobile device 2702, such as a smart phone that is used to input from the user the desired frequency and current level (or surgery mode, such as monopolar or bipolar) desired. This feature eliminates the need for an interface on the electrosurgical device 100 itself and thereby reduces the space required on the housing 200, as well as the overall size of the instrument.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A handheld and self-contained electrosurgical instrument, comprising:
a self-contained housing that is radio frequency permeable;
a plurality of rechargeable batteries located within the housing, the batteries for providing a direct current power source;
a microprocessor located within the housing and conductively coupled to the batteries, the microprocessor for receiving an external radio frequency signal that defines a selected frequency and current level;
a circuit located within the housing and conductively coupled to the microprocessor, the circuit for producing an RF signal according to said selected frequency and current level of a plurality of selectable frequency and current levels that, when applied to an electrode, performs at least one of cutting, coagulating and combined cutting-coagulating of biological tissue;
a radio frequency transformer located within the housing and conductively coupled to the circuit, the transformer for transforming the RF signal according to predefined attributes; and
an active monopolar or bipolar electrode located outside a distal end of the housing and conductively coupled to the transformer, the electrode for receiving the RF signal from the transformer and contacting biological tissue so as to cut and/or coagulate said biological tissue.

2. The electrosurgical instrument of claim 1, wherein the housing comprises at least two depressions near the distal end, wherein said depressions are designed to accept a user's fingers.

3. The electrosurgical instrument of claim 2, wherein the distal end of the housing including the electrode is removable.

4. The electrosurgical instrument of claim 3, wherein the housing is configured to allow the plurality of rechargeable batteries to be removed from the housing without the use of tools.

5. The electrosurgical instrument of claim 1, further including a RF receiver coupled to the microprocessor, the RF receiver for receiving the external radio frequency signal that defines the selected frequency and current level.

6. The electrosurgical instrument of claim 1, wherein the plurality of rechargeable batteries are arranged in a circular pattern within the housing.

7. The electrosurgical instrument of claim 1, further including a switch on an exterior of the housing for activating and deactivating the electrosurgical instrument.

8. A handheld radio frequency (RF) electrosurgical instrument adapted for performing electrosurgical procedures, the instrument comprising:
an elongated self-contained housing having at one end a terminal for receiving an electrosurgical electrode and having at least a portion that is permeable to wireless RF signals;
one or more rechargeable batteries located within the housing, the batteries for providing a direct current power source;
electronic circuitry located within the housing adjacent the portion that is permeable, the electronic circuitry powered by the power source and including at least a receiver for the wireless RF signals and a microprocessor connected to the receiver;
a first circuit in the electronic circuitry for generating a RF carrier wave including modulations which, when applied to the RF carrier wave, generate a modulated carrier wave that, when applied to an electrode, performs at least one of cutting, coagulating and combined cutting-coagulating of biological tissue;
a second circuit in the electronic circuitry for activating and deactivating the first and second circuits; and
an electrosurgical electrode directly mounted to the terminal and connected to receive the modulated carrier wave,
wherein the microprocessor, in response to signals received from the wireless receiver, is programmed to activate and deactivate the electronic circuitry and to select a desired modulation for the carrier wave and transmit the modulated carrier wave to the electrosurgical electrode.

9. The electrosurgical instrument of claim 8, wherein the housing comprises at least two depressions near the distal end, wherein said depressions are designed to accept a user's fingers.

10. The electrosurgical instrument of claim 9, wherein a distal end of the housing including the electrode is removable.

11. The electrosurgical instrument of claim 10, wherein the housing is configured to allow the plurality of rechargeable batteries to be removed from the housing without the use of tools.

12. The electrosurgical instrument of claim 8, wherein the rechargeable batteries are arranged in a circular pattern within the housing.

13. The electrosurgical instrument of claim 8, further including a switch on an exterior of the housing for activating and deactivating the electrosurgical instrument.

14. A system for performing electrosurgery, the system comprising:
a mobile device including a display for accepting input from a user and a radio frequency transmitter for transmitting a radio frequency signal that defines a mode; and
a handheld and self-contained electrosurgical instrument, comprising:
a self-contained housing that is radio frequency permeable and includes at a distal end a terminal for receiving a single electrosurgical electrode;
a plurality of rechargeable batteries located within the housing, the batteries for providing a direct current power source;
a microprocessor located within the housing and conductively coupled to the batteries, the microprocessor for receiving the radio frequency signal from the mobile device, wherein the mode defines a selected frequency and current level;
a circuit located within the housing and conductively coupled to the microprocessor, the circuit for producing an RF signal according to said selected frequency and current level of a plurality of selectable frequency and current levels that, when applied to an electrode, performs at least one of cutting, coagulating and combined cutting-coagulating of biological tissue;
a radio frequency transformer located within the housing and conductively coupled to the circuit, the transformer for transforming the RF signal according to predefined attributes;
a removable active monopolar electrode configured for connecting to the terminal of the housing and thereby conductively coupling to the transformer, the electrode for receiving the RF signal from the transformer and contacting biological tissue so as to cut and/or coagulate said biological tissue; and
a removable bipolar electrode configured for connecting to the terminal of the housing and thereby conductively coupling to the transformer, the electrode for receiving the RF signal from the transformer and contacting biological tissue so as to cut and/or coagulate said biological tissue.

15. The system of claim 14, wherein the housing comprises at least two depressions near the distal end, wherein said depressions are designed to accept a user's fingers.

16. The system of claim 15, wherein the housing is configured to allow the plurality of rechargeable batteries to be removed from the housing without the use of tools.

17. The system of claim 14, further including a RF receiver coupled to the microprocessor, the RF receiver for receiving the radio frequency signal from the mobile device that defines the selected frequency and current level.

18. The system of claim 14, wherein the plurality of rechargeable batteries are arranged in a circular pattern within the housing.

19. The system of claim 14, further including a switch on an exterior of the housing for activating and deactivating the electrosurgical instrument.

20. The system of claim 14, wherein the mode defines a selected modulation, frequency and current level and wherein the circuit produces an RF signal according to said selected modulation, frequency and current level of a plurality of selectable modulations, frequency and current levels that, when applied to an electrode, performs at least one of cutting, coagulating and combined cutting-coagulating of biological tissue.

21. A handheld and self-contained electrosurgical instrument, comprising:
a self-contained housing that is radio frequency permeable;
a plurality of rechargeable batteries located within the housing, the batteries for providing a direct current power source;
an interface coupled to the housing for accepting a selected frequency and current level from a user;
a microprocessor located within the housing and conductively coupled to the batteries, the microprocessor for reading the selected frequency and current level;
a circuit located within the housing and conductively coupled to the microprocessor, the circuit for producing an RF signal according to said selected frequency and current level of a plurality of selectable frequency and current levels that, when applied to an electrode, performs at least one of cutting, coagulating and combined cutting-coagulating of biological tissue;
a radio frequency transformer located within the housing and conductively coupled to the circuit, the transformer for transforming the RF signal according to predefined attributes; and
an active monopolar or bipolar electrode located outside a distal end of the housing and conductively coupled to the transformer, the electrode for receiving the RF signal from the transformer and contacting biological tissue so as to cut and/or coagulate said biological tissue.
